(19) United States
(12) Patent Application Publication
(10) Pub. No.: US 2019/0366330 A1
(43) Pub. Date: Dec. 5, 2019

(54) METHODS RELATING TO CRYOPRESERVATION

(71) Applicant: THE BRIGHAM AND WOMEN'S HOSPITAL, INC., Boston, MA (US)

(72) Inventors: Raymond Manohar ANCHAN, Sharon, MA (US); Sinan GUVEN, Izmir (TR); Utkan DEMIRCI, Cambridge, MA (US); George Luther MUTTER, Boston, MA (US)

(73) Assignee: THE BRIGHAM AND WOMEN'S HOSPITAL, INC., Boston, MA (US)

(21) Appl. No.: 16/535,740

(22) Filed: Aug. 8, 2019

Related U.S. Application Data
(62) Division of application No. 15/509,227, filed on Mar. 7, 2017, now abandoned.
(60) Provisional application No. 62/057,515, filed on Sep. 30, 2014.

(51) Int. Cl. B01L 3/00; A01N 1/02; G01N 1/42; A61K 35/12; C12N 1/04; C12N 5/073; C12N 5/071

(52) U.S. Cl. CPC B01L 3/5027; A01N 1/0221; G01N 1/42; A01N 1/0268; A01N 1/0284; B01L 2300/1894; C12N 1/04; C12N 5/0603; C12N 5/0672; B01L 2200/0652; B01L 2200/10; A61K 35/12

(57) ABSTRACT

The technology described herein is directed to methods of cryopreservation, e.g., cryopreservation in a microfluidics format and methods of utilizing cells preserved by such methods.

### CROSS-REFERENCE TO RELATED APPLICATIONS

This application is a divisional under 35 U.S.C. § 121 of co-pending U.S. application Ser. No. 15/509,227 filed Mar. 7, 2017, which is a 35 U.S.C. § 371 National Phase Entry Application of International Application No. PCT/US2015/053109 filed Sep. 30, 2015, which designates the U.S. and claims benefit under 35 U.S.C. § 119(e) of U.S. Provisional Application No. 62/057,515 filed Sep. 30, 2014, the contents of each of which are incorporated herein by reference in their entireties.

### GOVERNMENT SUPPORT

This invention was made with government support under Grant Nos. 5R01EB015776-02 awarded by the National Institutes of Health. The U.S. government has certain rights in the invention.

### TECHNICAL FIELD

The technology described herein relates to cryopreservation.

### BACKGROUND

Current clinical approaches to regenerative medicine aim to utilize pluripotent stem cells in cell- and gene-based therapies and tissue engineering applications. Existing tissue culture methods face specific challenges that include long-term viability of differentiated tissues in culture. For example, embryoid bodies (EBs) are formed from embryonic stem cells or induced pluripotent stem cells (iPSCs) and theoretically have the potential to differentiate into any desired cell type such as cardiac cells, osteogenic and chondrogenic cells, neurons, insulin secreting beta cells and steroid hormone secreting cells². EBs are three dimensional and thus their growth and duration of culture are restricted due to technical limitations such as penetration of media nutrients to the EB's core.

### SUMMARY

Described herein is an innovative culture system to grow, differentiate, and cryopreserve EBs in a microfluidic system that permits development of functionally specialized cells and tissues, such as ovarian cells and endocrine tissue. Notably, the systems and methods described herein thus permit the long-term storage of differentiated cells, e.g. EBs, in a microfluidic system. Thus, differentiated cells are available on-demand for therapeutic applications.

In one aspect, described herein is a method of cryopreserving a cell, the method comprising: contacting a cell with an isopropanol solution; and lowering the temperature of the cell and the solution to a temperature suitable for cryopreservation. In one aspect, described herein is a method of cryopreserving a cell, the method comprising: contacting a cell with an isopropanol solution; the solution being at a temperature suitable for cryopreservation.

In some embodiments of any of the aspects described herein, the cell is on a microfluidic device. In some embodiments of any of the aspects described herein, contacting the cell comprises flowing the isopropanol solution through the microfluidic device. In some embodiments of any of the aspects described herein, the method further comprises the step of sealing the microfluidic device following the contacting step.

In one aspect, described herein is a method of cryopreserving a cell, the method comprising: contacting a cell on a microfluidic device with a cryoprotectant solution; sealing the microfluidic device; contacting the sealed device with an isopropanol solution; and lowering the temperature of the solution to a temperature suitable for cryopreservation. In one aspect, described herein is a method of cryopreserving a cell, the method comprising: contacting a cell on a microfluidic device with a cryoprotectant solution; sealing the microfluidic device; and contacting the sealed device with an isopropanol solution the solution being at a temperature suitable for cryopreservation.

In some embodiments of any of the aspects described herein, the cell is a differentiated cell. In some embodiments of any of the aspects described herein, the cell is a cell differentiated in vitro. In some embodiments of any of the aspects described herein, the cell is an embryoid body cell. In some embodiments of any of the aspects described herein, the cell is a steroidogenic cell. In some embodiments of any of the aspects described herein, the cell is adhering to a surface.

In some embodiments of any of the aspects described herein, the isopropanol solution is at least 40% isopropanol. In some embodiments of any of the aspects described herein, the isopropanol solution is at least 50% isopropanol. In some embodiments of any of the aspects described herein, the isopropanol solution is at least 70% isopropanol. In some embodiments of any of the aspects described herein, the isopropanol solution is at least 80% isopropanol. In some embodiments of any of the aspects described herein, the isopropanol solution is at least 90% isopropanol. In some embodiments of any of the aspects described herein, the isopropanol solution is 100% isopropanol. In some embodiments of any of the aspects described herein, the isopropanol solution does not comprise DMSO. In some embodiments of any of the aspects described herein, the isopropanol solution does not comprise a cryoprotectant. In some embodiments of any of the aspects described herein, the cryoprotectant is selected from the group consisting of: DMSO; hydroxyethyl starch; glycerol; trehalose; polyethylene glycol; sucrose; dextrose; polyvinylpyrrolidone; methylcellulose; proline; a polymer; and ectoin.

In some embodiments of any of the aspects described herein, the cryoprotectant solution comprises from about 5% to about 50% DMSO. In some embodiments of any of the aspects described herein, the cryoprotectant solution comprises about 20% DMSO. In some embodiments of any of the aspects described herein, the cryoprotectant solution comprises DMSO and serum. In some embodiments of any of the aspects described herein, the cryoprotectant solution comprises from about 50% to about 95% serum. In some embodiments of any of the aspects described herein, the cryoprotectant solution comprises about 80% serum.

In some embodiments of any of the aspects described herein, the temperature suitable for cryopreservation is −60 C or lower. In some embodiments of any of the aspects described herein, the temperature suitable for cryopreservation is about −80 C or lower. In some embodiments of any of the aspects described herein, the method further comprises maintaining the cell at a temperature suitable for cryopreservation. In some embodiments of any of the aspects described herein, maintaining the cell at a temperature suitable for cryopreservation comprises keeping the cell and/or microfluidic device in liquid nitrogen. In some embodiments of any of the aspects described herein, the method further comprises thawing the cell and maintaining the cell in in vitro culture.

In one aspect, described herein is a method of providing a differentiated cell for treating a subject; the method comprising: obtaining a stem or progenitor cell from a first subject; differentiating the cell in vitro; cryopreserving the differentiated cell according to any of methods described herein; and thawing the differentiated cell. In some embodiments of any of the aspects described herein, the thawed cell is administered to a second subject. In some embodiments of any of the aspects described herein, the thawed cell is cultured in vitro and a cell product collected from the culture supernatant is administered to a second subject. In some embodiments of any of the aspects described herein, the cell product is a hormone or steroid hormone. In some embodiments of any of the aspects described herein, the hormone is selected from the group consisting of: estrogen; progesterone; or estradiol. In some embodiments of any of the aspects described herein, the cell product is dopamine or insulin. In some embodiments of any of the aspects described herein, the cell is cultured in vitro in a microfluidic device. In some embodiments of any of the aspects described herein, the first and second subjects are the same subject. In some embodiments of any of the aspects described herein, the differentiation occurs in a microfluidic device. In some embodiments of any of the aspects described herein, the cryopreservation occurs in a microfluidic device. In some embodiments of any of the aspects described herein, the thawing occurs in a microfluidic device. In some embodiments of any of the aspects described herein, the differentiated cell is an embryoid body cell. In some embodiments of any of the aspects described herein, the differentiated cell is a steroidogenic cell. In some embodiments of any of the aspects described herein, the differentiated cell is a beta-islet cell. In some embodiments of any of the aspects described herein, the stem cell is an iPSC.

### BRIEF DESCRIPTION OF THE DRAWINGS

FIG. 1 depicts a schematic of one embodiment of a sperm banking cassette as described herein.

FIG. 2 depicts a schematic diagram of the experimental setup of Example 4. Mouse embryonic stem cells are suspended in agarose-coated tissue culture dishes to generate embryoid bodies (EBs). Generated EBs are partially-embedded in Matrigel within a microfluidic channel. A constant and continuous, 2 μL/min flow of EB medium is flown in the channels for 21 days. Conditioned media is collected daily for ELISA analysis of hormone production (estradiol, testosterone, progesterone and AMH). Morphology of EBs after 21 days, viability of EBs through live-dead staining immunocytochemistry (ICC) for differentiation and proliferation assays were assessed at day 21.

FIGS. 3A-3C demonstrate that steroid hormones are secreted by mouse EBs in a microfluidic chip detected by ELISA analysis after 21 days of culture. Non-cryopreserved samples (black bars) and cryopreserved samples (white bars). FIG. 3A) estradiol, FIG. 3B) progesterone, FIG. 3C) testosterone.

### DETAILED DESCRIPTION

Described herein are methods for the cryopreservation of differentiated cells in, e.g., microfluidic systems, thereby permitting rapid provision of differentiated cells and/or their products for therapeutic purposes. This is a significant advantage over existing methods that require 1) preservation of stem or progenitor cells (thus requiring a long period of differentiation after thawing), 2) preservation of differentiated cells in formats that are not useful for therapeutic uses (requiring a long period of populating a therapeutically-useful format with the cells), or 3) use of non-preserved cells (limiting their use to a short time period before requiring a new population of cells).

Described herein are two methods of cryopreservation of cells, e.g. via direct contact with an isopropanol solution and by indirect contact preservation with an isopropanol solution. In some embodiments of the various aspects described herein, the isopropanol solution is provided at a temperature suitable for cryopreservation. In some embodiments of the various aspects described herein, the isopropanol solution is provided at a first temperature and lowered to a temperature suitable for cryopreservation after contacting the cells.

In one aspect, described herein is a method of cryopreserving a cell, the method comprising: contacting a cell directly with an isopropanol solution; and lowering the temperature of the cell and the solution to a temperature suitable for cryopreservation. In one aspect, described herein is a method of cryopreserving a cell, the method comprising: contacting a cell directly with an isopropanol solution; the solution being at a temperature suitable for cryopreservation.

In one aspect, described herein is a method of cryopreserving a cell, the method comprising: contacting a cell on a microfluidic device with a cryoprotectant solution; sealing the microfluidic device; contacting the sealed device with an isopropanol solution; and lowering the temperature of the solution to a temperature suitable for cryopreservation. In one aspect, described herein is a method of cryopreserving a cell, the method comprising: contacting a cell on a microfluidic device with a cryoprotectant solution; sealing the microfluidic device; and contacting the sealed device with an isopropanol solution the solution being at a temperature suitable for cryopreservation.

In some embodiments of any of the aspects described herein, the cell that is cryopreserved is a differentiated cell. In some embodiments of any of the aspects described herein, the cell that is cryopreserved is a cell differentiated in vitro. In some embodiments of any of the aspects described herein, the cell that is cryopreserved is a cell differentiated in vitro from a stem cell. In some embodiments of any of the aspects described herein, the cell that is cryopreserved is a cell differentiated in vitro from an induced pluriopotent stem cell (iPSC). In some embodiments of any of the aspects described herein, the cell that is cryopreserved is a cell differentiated in vitro from a progenitor cell. In some embodiments of any of the aspects described herein, the cell that is cryopreserved is an embryoid body cell. In some embodiments of any of the aspects described herein, an embryoid body is cryopreserved. In some embodiments of any of the aspects described herein, the cell that is cryopreserved is a steroidogenic cell. In some embodiments of any of the aspects described herein, the cell that is cryopreserved is a cell that is adhering to a surface.

As used herein “isopropanol solution” refers to a liquid comprising at least 40% isopropanol. In some embodiments of any of the aspects described herein, the isopropanol solution is at least 40% isopropanol. In some embodiments of any of the aspects described herein, the isopropanol solution is at least 50% isopropanol. In some embodiments of any of the aspects described herein, the isopropanol solution is at least 60% isopropanol. In some embodiments of any of the aspects described herein, the isopropanol solution is at least 70% isopropanol. In some embodiments of any of the aspects described herein, the isopropanol solution is at least 80% isopropanol. In some embodiments of any of the aspects described herein, the isopropanol solution is at least 90% isopropanol. In some embodiments of any of the aspects described herein, the isopropanol solution is at least 95% isopropanol. In some embodiments of any of the aspects described herein, the isopropanol solution is at least 98% isopropanol. In some embodiments of any of the aspects described herein, the isopropanol solution is 100% isopropanol. In some embodiments of any of the aspects described herein, the isopropanol solution consists essentially of isopropanol. In some embodiments of any of the aspects described herein, the isopropanol solution does not comprise a cyroprotectant. In some embodiments of any of the aspects described herein, the isopropanol solution does not comprise DMSO.

In some embodiments, the temperature of the isopropanol solution, e.g. either directly or indirectly in contact with the cells can be lowered over time. In some embodiments, the temperature of the isopropanol solution during the contacting step can be about the same temperature as the cells. In some embodiments, the temperature of the isopropanol solution during the contacting step can be about 30-40° C. In some embodiments, the temperature of the isopropanol solution during the contacting step can be about 20-30° C. In some embodiments, the temperature of the isopropanol solution during the contacting step can be about 10-20° C. In some embodiments, the temperature of the isopropanol solution during the contacting step can be about 0-10° C. In some embodiments, the temperature of the isopropanol solution during the contacting step can be about −10 to about 0° C. In some embodiments, the temperature of the isopropanol solution during the contacting step can be about −15 to about −5° C. In some embodiments, the temperature of the isopropanol solution during the contacting step can be about −20 to about −10° C.

In some embodiments, the temperature of the isoproponal solution can be lowered from about room temperature and/or about the temperature of the cells to about −80° C. by freezing the solution, and any cells and/or devices it is in contact with, at −80° C. for at least 30 minutes, e.g., at least 30 minutes, at least 1 hour, at least 2 hours, at least 4 hours, at least 6 hours, at least 12 hours, or longer. In some embodiments, the temperature of the isopropanol solution can be lowered by a slow-freezing protocol, e.g. as opposed to a vitrification protocol.

Protocols for cryopreservation, including details of vitrification and slow-freezing procedures and temperature changes are known in the art, see, e.g., Chian et la. Fertilit Cryopreservation 2010 Cambridge University Press; Simione “Thermo Scientific Nalgene and Nunc Cyropreservation Guide” 2009; “Cyropreservation” Biofiles Volume 5 No. 4 2010; each of which is incorporated by reference herein in its entirety.

As used herein “temperature suitable for cryopreservation” refers to a temperature that permits sustained cryopreservation, e.g. a temperature low enough to preserve viability without irreparably damaging the entire sample. The temperature of a cell and/or solution can be manipulated by a number of methods known in the art, e.g., a cooling bath, slow programmable freezing, a portable freezing container, a rate-controlled freezer, and vitrification. In some embodiments, the temperature suitable for cryopreservation is about −60° C. or lower. In some embodiments, the temperature suitable for cryopreservation is about −80° C. or lower. In some embodiments, the temperature suitable for cryopreservation is from about −60° C. to about −200° C.

In some embodiments of any of the aspects described herein, the cell is on or in a microfluidic device. In some embodiments of any of the aspects described herein, the cell is adhered to a surface of a microfluidic device. In some embodiments of any of the aspects described herein, the cell is growing in a layer of Matrigel™ on or in a microfluidic device. In some embodiments of any of the aspects described herein, the cell is growing in a layer of synthetic or natural extracellular matrix on or in a microfluidic device.

In some embodiments of any of the aspects described herein, contacting the cell comprises flowing the isopropanol solution through the microfluidic device. In some embodiments of any of the aspects described herein, contacting the cell comprises replacing growth medium or cell culture medium in the microfluidic device with the isopropanol solution, e.g. replacing at least 80%, at least 90%, at least 95%, at least 98% or more of the medium with the isopropanol solution. In some embodiments of any of the aspects described herein, the method can further comprise the step of sealing the microfluidic device following the contacting step, e.g. once the growth and/or culture medium is replaced by the isopropanol solution. The microfluidic device can be sealed by a number of means known in the art, including, by way of non-limiting examples, inserting a plug into a port, closing a valve, causing the device to fracture or break where a channel in the chip features self-sealing construction, or melting the chip at one or more points (e.g. by thermal or chemical means).

As used herein “cryoprotectant solution” refers to a mixture that is liquid at room temperature and which comprises at least one cyroprotectant. As used herein, “cryoprotectant” refers to a compound added to a biological sample in order to minimize or reduce the damage caused by freezing. Non-limiting examples of cryoprotectants can include DMSO; hydroxyethyl starch; glycerol; sugars; trehalose; polyethylene glycol; sucrose; dextrose; polyvinylpyrrolidone; methylcellulose; proline; a polymer; and ectoin. Cryoprotectants are known in the art and described further, e.g., in Janz et al. Journal of Biomedicine and Biotechnology 2012; Mareschi et al. Experimental Hematology 2006 34:1563-1572; and Hunt et al. Transfus Med Hemother 2011 38:107-123; each of which is incorporated by reference herein in its entirety.

In some embodiments of any of the aspects described herein, the cryoprotectant solution comprises from about 5% to about 50% cryoprotectant, e.g., DMSO. In some embodiments of any of the aspects described herein, the cryoprotectant solution comprises about 20% cryoprotectant, e.g., DMSO. In some embodiments of any of the aspects described herein, the cryoprotectant solution comprises cryoprotectant, e.g., DMSO, and growth medium (e.g., serum). In some embodiments of any of the aspects described herein, the cryoprotectant solution comprises from about 50% to about 95% growth medium (e.g. serum). In some embodiments of any of the aspects described herein, the cryoprotectant solution comprises about 80% growth medium (e.g. serum).

In some embodiments of any of the aspects described herein, the method can further comprise maintaining the cell at a temperature suitable for cryopreservation. In some embodiments of any of the aspects described herein, maintaining the cell at a temperature suitable for cryopreservation can comprise keeping the cell and/or microfluidic device in liquid nitrogen and/or in a freezer capable of maintaining a temperature suitable for cyropreservation.

In some embodiments of any of the aspects described herein, the method can further comprise thawing the cell and maintaining the cell in in vitro culture.

Cells cryopreserved according to the methods described herein can be utilized for therapeutic and/or screening purposes. In one aspect, described herein is a method of providing a differentiated cell for treating a subject; the method comprising: obtaining a stem or progenitor cell from a first subject; differentiating the cell in vitro; cryopreserving the differentiated cell according to any of the embodiments described herein; and thawing the differentiated cell.

In some embodiments of any of the aspects described herein, the thawed cell can be administered to a second subject. In some embodiments of any of the aspects described herein, the first and second subjects are the same subject, i.e. the differentiated cell is autologous to the subject receiving the treatment.

In some embodiments of any of the aspects described herein, the thawed cell can be cultured in vitro and a cell product collected from the culture supernatant administered to a second subject. The cell product can be any molecule released and/or secreted by the cell, e.g., a nucleic acid, polypeptide, or small molecule. In some embodiments of any of the aspects described herein, the thawed cell can be cultured in or on a microfluidic device used in the cryopreservation step, e.g., the cell is thawed and then cultured without removing it from the microfluidic device. In some embodiments of any of the aspects described herein, the culture supernatant can be collected from the outflow of the microfluidic device.

In some embodiments of any of the aspects described herein, the cell product is a hormone or steroid hormone. In some embodiments of any of the aspects described herein, the hormone is selected from the group consisting of: estrogen; progesterone; or estradiol. In some embodiments of any of the aspects described herein, the differentiated cell is an embryoid body cell. In some embodiments of any of the aspects described herein, the differentiated cell is a steroidogenic cell.

In some embodiments of any of the aspects described herein, the differentiated cell is a beta-islet cell. In some embodiments of any of the aspects described herein the cell product is dopamine or insulin.

In some embodiments of any of the aspects described herein the cell is cultured in vitro in a microfluidic device. In some embodiments of any of the aspects described herein, the differentiation occurs in a microfluidic device. In some embodiments of any of the aspects described herein, the cryopreservation occurs in a microfluidic device. In some embodiments of any of the aspects described herein, the thawing occurs in a microfluidic device.

In some embodiments of any of the aspects described herein, the stem cell is an iPSC. In some embodiments of any of the aspects described herein, the stem cell is an adult stem cell.

In one aspect, the methods described herein can relate to drug-screening. For example, in one aspect, described herein is a method comprising: obtaining a stem or progenitor cell from a first subject; differentiating the cell in vitro; cryopreserving the differentiated cell according to any of the embodiments described herein; thawing the differentiated cell; providing a test agent to the differentiated cell; and determining the effect of the test agent. In one aspect, described herein is a method comprising: obtaining a cell from a first subject; cryopreserving the cell according to any of the embodiments described herein; thawing the cell; providing a test agent to the cell; and determining the effect of the test agent. The test agent can be, e.g., an established medication (e.g. an FDA approved medication) for a condition the subject is in need of treatment for, e.g., the method can relate to finding an efficacious treatment and/or dosage regimen for that particular subject prior to the subject undergoing actual treatment. Alternatively, the test agent can be, e.g., an agent being screened for therapeutic activity for a condition, e.g. a condition the subject is in need of treatment for, e.g., the method can relate to finding an efficacious treatment for a treatment without necessarily comprising treatment of the subject themselves. In some embodiments of the foregoing aspects, the cell can be a diseased cell. In some embodiments of the foregoing aspects, the subject can be a subject with a disease. In some embodiments of the foregoing aspects, the cell can be a tumorigenic cell.

The compositions and methods described herein can be administered to a subject having or diagnosed as having a condition or disease. In some embodiments, the methods described herein comprise administering an effective amount of compositions described herein, e.g. cell products to a subject in order to alleviate a symptom of a condition or disease. As used herein, “alleviating a symptom” is ameliorating any condition or symptom associated with the disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of a composition (e.g. cells and/or cell products) needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular therapeutic effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

In some embodiments, the technology described herein relates to a pharmaceutical composition comprising a cell or cell product as described herein, and optionally a pharmaceutically acceptable carrier. In some embodiments, the active ingredients of the pharmaceutical composition comprise a cell or cell product as described herein. In some embodiments, the active ingredients of the pharmaceutical composition consist essentially of a cell or cell product as described herein. In some embodiments, the active ingredients of the pharmaceutical composition consist of cell or cell product as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent as described herein.

In some embodiments, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a composition as disclosed herein can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5674,533; 5,059,595; 5,591 ,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering a second agent and/or treatment to the subject, e.g. as part of a combinatorial therapy.

In certain embodiments, an effective dose of a composition comprising a cell or cell product as described herein can be administered to a patient once. In certain embodiments, an effective dose of a composition comprising a cell or cell product can be administered to a patient repeatedly. For systemic administration, subjects can be administered a therapeutic amount of a composition comprising a cell product, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising a cell or cell product as described herein can be administered over a period of time, such as over a 5 minute, 10 minute, 15 minute, 20 minute, or 25 minute period.

The dosage ranges for the administration of a composition according to the methods described herein depend upon, for example, the form of the active ingredient, its potency, and the extent to which symptoms, markers, or indicators of a condition described herein are desired to be reduced, for example the percentage reduction desired for a symptom. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition in, e.g. the treatment of a condition as described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. hormone levels. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of hormone deficiencies. When using an experimental animal model, efficacy of treatment is evidenced when a statistically significant change in a marker is observed, e.g. hormone levels.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a disease or condition. A subject can be male or female.

As used herein, “contacting” refers to any suitable means for delivering, or exposing, an agent (e.g. an isopropanol solution or a cryoprotectant) to at least one cell or device. Exemplary delivery methods include, but are not limited to, direct delivery to cell culture medium, perfusion, injection, submersion, or other delivery method well known to one skilled in the art.

As used herein, “cryopreservation” refers to the cooling and storing of biological samples, e.g. cells or tissues, at very low temperatures to maintain their viability.

As used herein, the term “stem cell” refers to a cell in an undifferentiated or partially differentiated state that has the property of self-renewal and has the developmental potential to naturally differentiate into a more differentiated cell type, without a specific implied meaning regarding developmental potential (i.e., totipotent, pluripotent, multipotent, etc.). By self-renewal is meant that a stem cell is capable of proliferation and giving rise to more such stem cells, while maintaining its developmental potential. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells and hematopoietic stem cells. In some embodiments, the stem or progenitor cells can be embryonic stem cells. As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are totipotent cells derived from the early embryo or blastocyst. Embryonic stem cells can be obtained directly from suitable tissue, including, but not limited to human tissue, or from established embryonic cell lines. In one embodiment, embryonic stem cells are obtained as described by Thomson et al. (U.S. Pat. Nos. 5,843,780 and 6,200,806; Science 282:1145, 1998; Curr. Top. Dev. Biol. 38:133 ff, 1998; Proc. Natl. Acad. Sci. U.S.A. 92:7844, 1995 which are incorporated by reference herein in their entirety).

Exemplary stem cells include induced pluriopotent stem cells, embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Descriptions of stem cells, including method for isolating and culturing them, may be found in, among other places, Embryonic Stem Cells, Methods and Protocols, Turksen, ed., Humana Press, 2002; Weisman et al., Annu. Rev. Cell. Dev. Biol. 17:387 403; Pittinger et al., Science, 284:143 47, 1999; Animal Cell Culture, Masters, ed., Oxford University Press, 2000; Jackson et al., PNAS 96(25):14482 86, 1999; Zuk et al., Tissue Engineering, 7:211 228, 2001 (“Zuk et al.”); Atala et al., particularly Chapters 33 41; and U.S. Pat. Nos. 5,559,022, 5,672,346 and 5,827,735.

As used herein, “progenitor cells” refers to cells in an undifferentiated or partially differentiated state and that have the developmental potential to differentiate into at least one more differentiated phenotype, without a specific implied meaning regarding developmental potential (i.e., totipotent, pluripotent, multipotent, etc.) and that does not have the property of self-renewal. Accordingly, the term “progenitor cell” refers to any subset of cells that have the developmental potential, under particular circumstances, to differentiate to a more specialized or differentiated phenotype. In some embodiments, the stem or progenitor cells are pluripotent stem cells. In some embodiments, the stem or progenitor cells are totipotent stem cells.

As used herein, a “differentiated cell” refers to a cell that is more specialized in its fate or function than at a previous point in its development, and includes both cells that are terminally differentiated and cells that, although not terminally differentiated, are more specialized than at a previous point in their development. The development of a cell from an uncommitted cell (for example, a stem cell), to a cell with an increasing degree of commitment to a particular differentiated cell type, and finally to a terminally differentiated cell is known as progressive differentiation or progressive commitment. In the context of cell ontogeny, the adjective “differentiated”, or “differentiating” is a relative term. A “differentiated cell” is a cell that has progressed further down the developmental pathway than the cell it is being compared with.

As used herein, the term “microfluidic device” refers to a structure or substrate having microfluidic structures contained therein or thereon. In some embodiments, the device can be detachably connected to a microfluidic system.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder associated with a condition. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of cryopreserving a cell, the method comprising:
   contacting a cell with an isopropanol solution; and lowering the temperature of the cell and the solution to a temperature suitable for cryopreservation.
2. A method of cryopreserving a cell, the method comprising:
   contacting a cell with an isopropanol solution; the solution being at a temperature suitable for cryopreservation.
3. The method of any of paragraphs 1-2, wherein the cell is on a microfluidic device.
4. The method of paragraph 3, wherein contacting the cell comprises flowing the isopropanol solution through the microfluidic device.
5. The method of any of paragraphs 2-4, further comprising the step of sealing the microfluidic device following the contacting step.
6. A method of cryopreserving a cell, the method comprising:
   contacting a cell on a microfluidic device with a cryoprotectant solution;
   sealing the microfluidic device;
   contacting the sealed device with an isopropanol solution; and
   lowering the temperature of the solution to a temperature suitable for cryopreservation.
7. A method of cryopreserving a cell, the method comprising:
   contacting a cell on a microfluidic device with a cryoprotectant solution;
   sealing the microfluidic device; and
   contacting the sealed device with an isopropanol solution the solution being at a temperature suitable for cryopreservation.
8. The method of any of paragraphs 1-7, wherein the cell is a differentiated cell.
9. The method of any of paragraphs 1-8, wherein the cell is a cell differentiated in vitro.
10. The method of any of paragraphs 1-9, wherein the cell is an embryoid body cell.
11. The method of any of paragraphs 1-10, wherein the cell is a steroidogenic cell.
12. The method of any of paragraphs 1-11, wherein the cell is adhering to a surface.
13. The method of any of paragraphs 1-12, wherein the isopropanol solution is at least 40% isopropanol.
14. The method of any of paragraphs 1-13, wherein the isopropanol solution is at least 50% isopropanol.
15. The method of any of paragraphs 1-14, wherein the isopropanol solution is at least 70% isopropanol.
16. The method of any of paragraphs 1-15, wherein the isopropanol solution is at least 80% isopropanol.
17. The method of any of paragraphs 1-16, wherein the isopropanol solution is at least 90% isopropanol.
18. The method of any of paragraphs 1-17, wherein the isopropanol solution is 100% isopropanol.
19. The method of any of paragraphs 1-18, wherein the isopropanol solution does not comprise DMSO.
20. The method of any of paragraphs 1-19, wherein the isopropanol solution does not comprise a cryoprotectant.
21. The method of paragraph 20, wherein the cryoprotectant is selected from the group consisting of:
   DMSO; hydroxyethyl starch; glycerol; trehalose; polyethylene glycol; sucrose; dextrose; polyvinylpyrrolidone; methylcellulose; proline; a polymer; and ectoin.
22. The method of any of paragraphs 1-21, wherein the cryoprotectant solution comprises from about 5% to about 50% DMSO.
23. The method of any of paragraphs 1-22, wherein the cryoprotectant solution comprises about 20% DMSO.
24. The method of any of paragraphs 1-23, wherein the cryoprotectant solution comprises DMSO and serum.
25. The method of any of paragraphs 1-24, wherein the cryoprotectant solution comprises from about 50% to about 95% serum.
26. The method of any of paragraphs 1-25, wherein the cryoprotectant solution comprises about 80% serum.
27. The method of any of paragraphs 1-26, wherein the temperature suitable for cryopreservation is −60 C or lower.
28. The method of any of paragraphs 1-27, wherein the temperature suitable for cryopreservation is about −80 C or lower.
29. The method of any of paragraphs 1-28, further comprising maintaining the cell at a temperature suitable for cryopreservation.
30. The method of paragraph 29, wherein maintaining the cell at a temperature suitable for cryopreservation comprises keeping the cell and/or microfluidic device in liquid nitrogen.
31. The method of any of paragraphs 1-39, further comprising thawing the cell and maintaining the cell in in vitro culture.
32. A method of providing a differentiated cell for treating a subject; the method comprising:
   obtaining a stem or progenitor cell from a first subject;
   differentiating the cell in vitro;
   cryopreserving the differentiated cell according to any of paragraphs 1-31; and
   thawing the differentiated cell.
33. The method of paragraph 32, wherein the thawed cell is administered to a second subject.
34. The method of paragraph 32, wherein the thawed cell is cultured in vitro and a cell product collected from the culture supernatant is administered to a second subject.
35. The method of paragraph 34, wherein the cell product is a hormone or steroid hormone.
36. The method of paragraph 35, wherein the hormone is selected from the group consisting of:
   estrogen; progesterone; or estradiol.
37. The method of paragraph 34, wherein the cell product is dopamine or insulin.
38. The method of any of paragraphs 32-37, wherein the cell is cultured in vitro in a microfluidic device.
39. The method of any of paragraphs 32-38, wherein the first and second subjects are the same subject.
40. The method of any of paragraphs 32-39, wherein the differentiation occurs in a microfluidic device.
41. The method of any of paragraphs 32-40, wherein the cryopreservation occurs in a microfluidic device.
42. The method of any of paragraphs 32-41, wherein the thawing occurs in a microfluidic device.
43. The method of any of paragraphs 32-42, wherein the differentiated cell is an embryoid body cell.
44. The method of any of paragraphs 32-43, wherein the differentiated cell is a steroidogenic cell.
45. The method of any of paragraphs 32-44, wherein the differentiated cell is a beta-islet cell.
46. The method of any of paragraphs 32-45, wherein the stem cell is an iPSC.

### EXAMPLES

### Example 1

Described herein is the employment of microfluidic cassettes as a novel platform for long-term culture and cryopreservation of functional, differentiated mouse embryoid bodies.

Materials and Methods: Embryoid bodies (EBs), grown in suspension from mouse embryonic stem cells (ESCs), were embedded in Matrigel-coated channels with a constant 1 μl/min flow of culture media for 21 days. EB viability, differentiation, and functionality were assayed as measures of the culture system's efficacy. Viability was assessed with Live/Dead stains and BrdU proliferation assays. Differentiation was analyzed with immunocytochemistry (ICC) for markers of endoderm, ectoderm, and mesoderm, as well as ovarian tissue. Hormone synthesis served as an indicator of EB functionality. Conditioned media collected over each 24-hour period was assayed by ELISA for estradiol (E2), progesterone, and testosterone synthesis. We also slow-froze sealed cassettes in isopropanol, thawed these, and repeated viability and functionality tests.

It is demonstrated herein that EBs grown in microfluidic cassettes maintain long-term viability and proliferation after 21 days.

Differentiation of EBs in the microfluidic system was verified, as shown by ICC of cell markers from all three germ layers and expression of ovarian cell markers (inhibin, Cyp19a1, and AMHR). Functional analysis shows increasing synthesis of E2 (15 pg/ml on Day 1 to 31 pg/ml on Day 20). Cryopreserved EB-laden microfluidic chips recovered upon thawing and continued hormone synthesis.

Microfluidic culture of functional EBs is a promising system that can maintain EB viability, differentiation, and functionality, even after recovery from cryopreservation and afford an opportunity to develop patient-specific cassettes of differentiated human ESCs that may be stored, used in drug testing, or harvested for hormones.

### Example 2

### Sperm Application on Microfluidic Chips and Functional Freezing of Cells on Microfluidic Chips

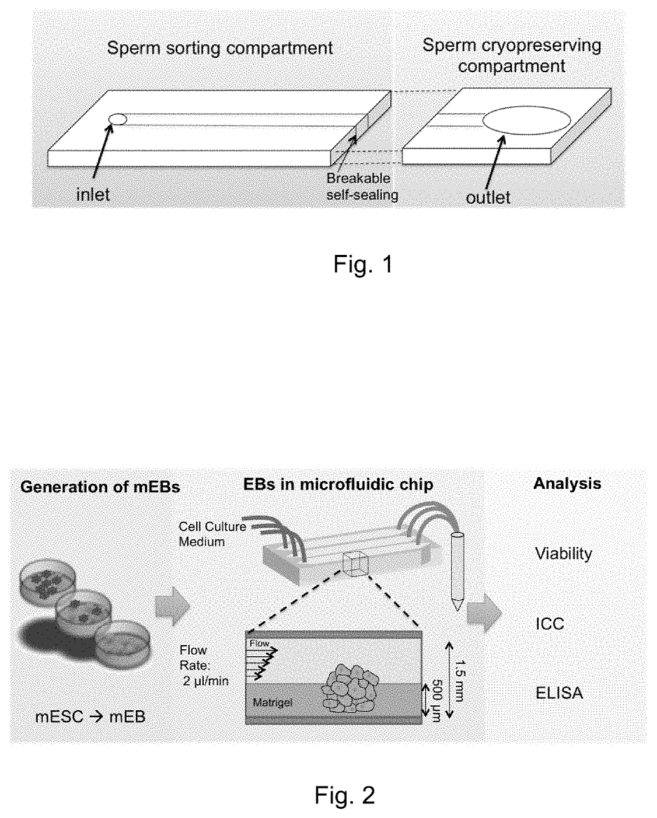

An advantage and application of sperm freezing on patient-specific microfluidic sperm banking cassettes (SBCs) relates to advancements in the technology of cryobiology and reproductive medicine. For the selection of the most potent sperm cells for a patient's in vitro fertilization treatment cycle at clinical embryology laboratories, one can utilize the invented SBCs. The total sperm sample is loaded into the microfluidic channels and followed by immediate sperm sorting for clinical use. This selection of the most viable sperm may be done in advance of a treatment cycle and sorted sperms can be easily cryopreserved within the same microcassette closed sterile environment in liquid nitrogen for banking for use on demand (FIG. 1).

Usually in cell banks and embryology laboratories the current procedures involve long and laborious steps. First of all, sperm sorting under current standard protocols requires processing of the raw sample through multiple centrifugation steps and followed by transfer of the sorted cells for cryopreservation. The invented SBC device not only selects the most motile sperm, but also provides the platform for cryopreservation in a cost and labor effective manner negating the need to transfer to another cryopreservation container. This helps minimize error and damage to the sample with minimal handling since each SBC is a patient-specific dedicated self-containing system for sorting and cryopreservation.

The use of these microfluidic chips to sort sperm as well as functioning as a closed system for freezing sperm is a significant novel advancement in approaches to cryo-banking of sperm. Furthermore, this advancement significantly decrease the number of intermediate steps currently required to achieve this process and minimizes damage to the sample.

In some embodiments, the microfluidic chips can be built in compartments where the sperm that reach the end of the channels are sorted. The final compartment with the sorted sperm can be broken and then used as a frozen vial for banking. These cells will be ready to use and already presorted and separated from the seminal fluid after thawing.

### Example 3

The potential for the use of embryonic and pluripotent stem cells in cell-based and regenerative therapies continues to be explored by better understanding specific culture conditions and differentiation signals to direct development of stem cells into desired tissue types. Embryoid bodies (EBs) are aggregates of differentiating stem cells that contain tissues from all three developmental germ layers and theoretically could generate every cell type in the body. EBs under specific culture conditions develop steroidogenic capacity. While long-term availability of steroidogenic stem cells would necessitate the repeated generation and culture of EBs, an arduous and time consuming process, described herein are methods and compositions for growing and developing functional EBs in microfluidic chips, permitting a personalized patient specific treatment cassette that is possible to cryopreserve until required for treatment use.

The microfluidic devices were designed and fabricated using 1.5 mm thick Poly(methyl methacrylate) (PMMA). Three 4 mm×28 mm parallel channels separated by a gap of 3 mm were cut onto a 24 mm×40 mm PMMA using a laser. A 24 mm×40 mm coverslip and double side adhesive film were used as the base and the middle layer of the microfluidic device respectively. A PMMA chip with 6 openings of 0.78 mm in diameter each was cut to serve as a top layer of the microfluidic device. Approximately 5×10Ep×6 EB cells/mL were mixed uniformly with Matrigel and applied in each microfluidic channel. Silicon tubes (inner diameter 0.25 mm) were inserted into the inlet and outlet openings for unidirectional flow through the microchannels. The microchip is supplied with continuous flow of fresh EB media at the rate of 2 μL/min. The terminal end of channels were connected to 15 ml centrifuge tubes collecting the drained conditioned media of 24 h at day 1, 5, 11, 15 and 21 for detection and quantification of secreted steroid hormones with ELISA.

As described herein, the system:
is able to keep the long term viability of embryoid bodies under continuous flow
can be cryopreserved and retain functionality after thaw
can synthesize bioidentical, autologous endocrine hormones
can also be used to freeze sperm cells after sorting in microfludic channels.
   This patient-specific personalized microfluidic cassete concept can be applied to other applications such as for example the generation and maintenance of insulin secreting cells or dopamine producing cells.

The systems described herein can be cryopreserved and fully function upon thawing on demand. Using autologous cells the system can be used to synthesize autologus reproductive endocrinal hormones toward personalized medicine.

The methods and compositions described herein permit the maintainence of stem cell-derivative endocrine tissue using patient-specific autologous cells.

### Example 4

### Functional Maintenance of Differentiated Embryoid Bodies in Microfluidic Systems: A Platform for Personalized Medicine

Hormone replacement therapies have become important for treating diseases such as premature ovarian failure or menopausal complications. The clinical use of bioidentical hormones may significantly reduce some of the potential risks reportedly associated with the use of synthetic hormones. Demonstrated herein is the utility and advantage of a microfluidic chip culture system to enhance the development of personalized, on demand-treatment modules using embryoid bodies (EBs). Functional EBs cultured on microfluidic chips represents a platform for personalized, patient-specific treatment cassettes that can be cryopreserved until required for treatment. We assessed the viability, differentiation, and functionality of EBs cultured and cryopreserved in this system. During extended microfluidic culture, estradiol, progesterone, testosterone and anti-Müllerian hormone levels were measured and the expression of differentiated steroidogenic cells was confirmed by immunocytochemistry assay for the ovarian tissue markers, anti-Müllerian hormone receptor type-II, follicle-stimulating hormone receptor, inhibin B and the estrogen biosynthesis enzyme aromatase. These studies demonstrated that under microfluidic conditions, differentiated steroidogenic EBs continued to secrete estradiol and progesterone at physiologically-relevant concentrations (30-120 pg/mL, 150-450 pg/mL respectively), for up to 21 days. Collectively, we demonstrate for the first time, the feasibility of using a microfluidic chip system with continuous flow for the differentiation and extended culture of functional steroidogenic stem cell-derived EBs, the differentiation of EBs into cells expressing ovarian antigens in a microfluidic system and the ability to cryopreserve this systems with restoration of growth and functionality upon thaw. These results present a platform to the development of a new therapeutic system for personalized medicine.

### Introduction

Ovaries have two distinct functions that are critical to a woman's reproductive health: hormone synthesis and gametogenesis. There is a significant population of reproductive-age patients who experience premature ovarian failure (POF) and lose regular hormone synthesis due to either iatrogenic causes, such as chemotherapy or idiopathic, presumably genetic causes. The number of female cancers diagnosed in reproductive age women is approaching 9% of all diagnoses¹ and survival will continue to climb as treatment options and novel biotechnological advances emerge². The loss of ovarian function has physiologic as well as considerable psychosocial repercussions on patients that negatively affect quality of life. Currently gonadal failure and the associated loss of hormone synthesis in patients with POF, or menopausal women, is treated by hormone replacement therapy (HRT) using synthetically-produced steroids'. However, the Women's Health Initiative (WHI) raised several outcome concerns related to this approach for two specific types of conjugated estrogens of hormones, Premarin® and Prempro®, which increased risk of stroke, blood clot, myocardial infarction and neoplasias⁴⁻¹¹. These reported observations have since been clinically expanded by health care providers to include all synthetically-generated hormones used in HRT. By contrast, recent reports suggest that bioidentical hormones may be a safer alternative for HRT¹⁰. The presumed risks associated with the current HRT treatment regimen necessitate improved therapeutic options. Described herein is a novel approach for HRT, using stem cells in a cell-based therapy. The data provided herein support the use of microfluidics as an opportunity for developing novel personalized medicine applications¹².

The pluripotent nature of embryonic stem cells (ESC) presents a unique opportunity for both researchers and clinicians to be able to generate any cell or tissue type through directed differentiation protocols. Non-directed differentiation of ESCs seeded on non-adhesive plates is in suspension, however, can lead to formation of an embryoid body (EB), a densely packed spheroid of embryonic stem cells that differentiate into cell types from all three developmental germ layers: endoderm, ectoderm, and mesoderm. More recent studies in our laboratory suggest that EBs derived from G4 mouse ESCs may differentiate under specific culture conditions into ovarian tissue, a primary steroidogenic organ of the female reproductive system¹³, and that these differentiated G4 EBs synthesize physiologically-relevant levels of estradiol¹⁴. Estradiol is the primary female hormone, important for women's health and development, and is used in a wide range of medical treatments, particularly in postmenopausal women and infertility patients.

Limitations of long term in vitro culture of EBs for therapeutic purposes using the current standard tissue culture approaches include the high cost, risk of contamination, dependency on the operator, labor intensity, and the necessity of large volumes of reagents. For example, during the interval between culture media changes, toxins and waste accumulation as well as depletion of nutrients may interfere with the metabolism of the EBs. Moreover with the increasing size of cultured EBs, we encounter the concern for insufficient gas and nutrient exchange at the core regions of the EB, which in turn may result in cell death within the EB inner mass¹⁵. By developing a system with continuous flow of fresh media, this limitation is addressed. By employing a dynamic continuous flow system of microfluidic chips not only the accumulation of toxins and waste is decreased but also it allows improved control of culture parameters, enabling standardized microenvironments and sustainable supply of fresh nutrients within a closed system in experiments¹⁶⁻¹⁹. Described herein is a method where EBs are immobilized in a closed microfluidic system that provides fresh media, while simultaneously collecting the steroid hormone from the supernatant from the terminal port. Using this approach, cells can be kept in a contained system and survive prolonged culture durations without requiring exposure to air or other sources of contamination. Furthermore, the differentiated EBs in individual chips can be cryopreserved and thawed on demand at a later time.

### Materials and Methods

### Generation of Embryoid Bodies (EBs)

Mouse embryonic fibroblast (MEF) medium was prepared by using DMEM supplemented with 10% heat-inactivated Fetal Bovine Serum (FBS) and 1% L-glutamine 200 mM (100×) (Life Technologies). 5×10⁵ MEF feeder cells were mitotically-inactivated using Mitomycin C (Sigma, St. Louis, Mo.) and seeded on a 100 mm tissue culture plate coated with 0.1% gelatin (Sigma, St. Louis, Mo.) in MEF medium. Cell culture plates were washed with phosphate buffer saline (PBS) (Life Technologies) solution and the media was changed every 2-3 days until cell were 75-80% confluent.

Mouse embryonic stem cell media (ES medium) was prepared using DMEM supplemented with 10% stem-cell grade FBS, 1% L-glutamine 200 mM (100×), 10⁵ units/L ESGRO mLIF (Millipore, Temecula, Calif.) and 0.2 mM 2-Mercaptoethanol (Sigma, St. Louis, Mo.). Approximately 2-4 hours before plating the G4 mouse embryonic stem cells (mESC) (Samuel Lunenfeld Research Institute, Toronto, Canada) onto the layer of MEF feeder cells, MEF medium was replaced with ES medium. 1×10⁶ mESCs were seeded on top of the feeder layer using ES medium. The media was changed every day for 5 days to obtain satisfactory amount of proliferating mESC colonies.

Mouse EB medium was prepared by using DMEM/F12 (1:1) 1× (Life Technologies) supplemented with 15% FBS, 15% Knock Out Serum (Life Technologies), 1% MEM Non-Essential Amino Acids 100× (Life Technologies), 1% of L-glutamine 200 mM (100×), 0.2 mM 2-Mercaptoethanol and 5 ng/ml of Basic Fibroblast Growth Factor (FGF-2; R&D Systems). 2×10⁶ mESCs were seeded on a 100 mm petri dish or 96 well plates coated with 1.5% agarose to generate EBs in a low-adhesion environment. By simple decantation method, at least 50% of the medium was replaced with fresh EB medium every day.

### Microfluidic Chip Fabrication

The microfluidic devices were designed and fabricated using 1.5 mm thick Poly(methyl methacrylate) (PMMA; McMaster Carr, Atlanta, Ga.) and 80 μm thick double-sided adhesive film (DSA) (iTapestore, Scotch Plains, N.J.) as described in previous studies¹⁶. Briefly, three 4 mm×28 mm parallel channels separated by a gap of 3 mm were cut onto a 24 mm×40 mm DSA film and PMMA plate using a laser cutter (Versa Laser™, Scottsdale, Ariz.). Surface of 24 mm×40 mm glass coverslip (150 μm thick) or Polystyrene plate (1 mm thick) was plasma treated for 90 sec and adhered to DSA film forming the base and the middle layer of the microfluidic device respectively. A 24 mm×40 mm PMMA chip with 3 inlet and 3 outlet openings of 0.78 mm in diameter (each was cut to serve as a top layer of the microfluidic device). The openings in this layer were aligned to the end point of the DSA channels to be used as inlets and outlets during the fluid flow. Finally, PMMA channels with inlet and outlet opening were assembled into the DSA-Polystyrene plate combination to make a three-layered microfluidic device with microchannels of 4 mm×28 mm×1.5 mm in dimension. All components used in assembly were cleaned with detergent, ethanol and UV sterilized for 15 min respectively under a laminar flow hood before assembly.

### Dynamic Culture of EBs in Microfluidic Chip

Approximately 5×10⁶ EB cells/mL were mixed uniformly with ice cold Matrigel® (Growth factor reduced, BD Biosciences). 70-100 μL of this EB-Matrigel® mixture was carefully pipetted into each 4 mm×28 mm×1.5 mm channel of the microfluidic chip. The assembled, cell-laden microfluidic chip was then transferred to 37° C. incubator for 15 minutes to produce a uniform layer of hydrogel upon gelation. After the gelation of Matrigel®, the third layer of the microchip (PMMA layer with the inlet and outlet openings) was carefully aligned and assembled onto body of the chip. Silicon tubes (inner diameter 0.25 mm) (Cole-Parmer, IL, Cat: EW-06419-00) were inserted into the inlet and outlet openings for unidirectional flow through the microchannels. The microchip with encapsulated EB cells was transferred into the cell culture incubator providing continuous flow of fresh EB media at the rate of 2 μL/min using 10 mL syringes (BD, Franklin, N.J.) and a syringe pump NE-1600 (New Era Pump Systems, Farmingdale, N.Y.). The terminal end of channels were connected to 15 ml tubes collecting the drained conditioned medium of 24 h at day 1, 5, 11, 15 and 21 for detection and quantification of secreted steroid hormones with ELISA.

### Cryopreservation of EB Immobilized Microfluidic Chips

After 24 h of dynamic culture EB immobilized microfluidic chips were washed with PBS and channels were filled with cryoprotecting solution (80% FBS, 20% dimethylsulfoxide). After blocking inlets and outlets microfluidic chips were sealed and immersed in isopropanol (Sigma) and frozen at −80° C. for overnight, then transferred into liquid nitrogen. After 48 h cryopreserved chips were thawed in 37° C. water bath and rinsed 3 times with fresh culture media.

### Viability and Proliferation Assays

The viability of cells within the EB was assessed after 21 days of microfluidic chip culture and after thawing with Calcein-AM/Ethidium homodimer-1, Live-Dead assay (Life Technologies). The assay was performed directly within the microfluidic chip without harvesting the EBs by incorporating Live-Dead kit reagents and subsequent washing steps. Samples were imaged with Zeiss Axio fluorescence microscope. The proliferation of cells was determined with BrdU proliferation assay kit (Sigma) according to manufacturer's instructions.

### Immunocytochemical Analysis

Mouse ESC colonies, EBs in suspension and EBs in microfluidic chip were harvested and fixed with 1% paraformaldehyde (Electron Microscopy Sciences, USA). The samples were blocked with 1% BSA (Sigma), permeabilized with 0.3% TritonX 100 (Sigma) and stained for stem cell markers, Oct-4 (Abcam: ab18976), SSEA-4 (Biolegend: 330410) and Nanog (Abcam: ab80892), germ layer markers alpha-fetoprotein (Santa Cruz Biotechnology: sc-8108), smooth muscle actin (Abcam: ab5694), and neurofilament (Abcam: ab7794) and ovarian tissue markers AMHR2 (Abcam: ab64762), inhibin β-A (Santa Cruz Biotechnology: sc-166503), FSHR (Santa Cruz Biotechnology: sc-7798 and Anti-Aromatase (CYP19A1) (Abcam: ab35604) primary antibodies overnight at 4° C. Alexa Fluor™ 488 and Alexa Fluor™ 568 were used as secondary antibody, cell nuclei were stained with DAPI (Life Technologies). Stained samples were analyzed with Zeiss LSM 510 META™ confocal microscope.

### Enzyme-Linked ImmunoSorbent Assay (ELISA)

Conditioned medium both from EBs cultured in 96 well plate under static condition and conditioned medium collected from terminal end of the EB immobilized microfluidic channels were collected for 24 hours period and analyzed for the presence of the sex hormones; estradiol, progesterone, and testosterone. Levels of secreted steroid hormones were detected by enzyme-linked immunosorbent assay (ELISA) using a specific kit for estradiol, progesterone and testosterone according to protocols of the Wisconsin National Primate Research center, University of Wisconsin-Madison. The antibody for estradiol has been supplied from Holly Hill Biologicals (Oregon, USA).

### Statistical Analyses

The experimental results were analyzed using ANOVA™ with Tukey's post hoc test for multiple comparisons and Student's two-tailed t test for single comparisons with statistical significance threshold set at 0.05 (P<0.05). Unless otherwise stated, mean values represent three experiments with two or three channels per experiment, and error bars represent standard error of the mean. Statistical analyses were performed with GraphPad Prism5™ (GraphPad).

### Results

A microfluidic device was fabricated to physically stimulate the generated EBs with continuous laminar flow and shear stress. Dynamic culture introduces mechanical stimulation on cells in their native environment¹⁶. The bottom of the device is designed as a 150 μm thick glass cover slip enabling sufficient penetration depth for monitoring the EBs with confocal microscopy. To immobilize the EBs within a microfluidic channel and provide ECM like support the EBs were plated within Matrigel® depth of 500 μm avoiding a total encapsulation. After immobilization of EBs the microfluidic channel allowed 1.5 mm of depth for the flow of the media (FIG. 2). Cell culture media was perfused with a syringe pump with flow rate of 2 μl/min. Silicon tubing was utilized, permitting gas exchange for the oxygenation of the media. The contained microfluidic system developed in this study provides advantages over classic 2D culture utilizing fewer amounts of reagents and multiplying the test conditions for high throughput analyses. Designed chip also allows in situ tracking and staining platform without the removal of the EBs from the channels.

### Microfluidics Supports the Long-Term Culture of Mouse ESC-Derived Embryoid Bodies

In this study embryoid bodies (EBs) were generated from mouse embryonic stem cells and incorporated into microfluidic channels and cultured under continuous flow (FIG. 2). The generated EBs range between 70-200 μm in diameter. After culture, under continuous laminar flow in microfluidic channels for 21 days the EBs were highly viable (data not shown) comparable to that observed in standard tissue culture plates. Minimal necrotic core was detected within the EBs (data not shown) demonstrating the microenvironment and the physiological conditions are supporting the viability of the cells. Also demonstrated was the preservation of metabolic activity of cells within the microfluidic culture. The proliferation of long term cultured EBs was investigated with BrdU assay. The newly formed cells within the EBs were detected with anti-BrdU assay (data not shown) showing that the cells are metabolically active and pursue proliferation.

mESCs and EBs Cultured in Microfluidic Chips Continue to Grow and Differentiate

Characterization of germ layers within EBs after static and microfluidic culture was assessed with immunocytochemistry for stem cell markers together with germ layer specific cell surface markers. Stemness properties of mESC colonies, EBs grown in static conditions and also microfluidic chips were assessed staining for anti-Nanog, anti-Oct-4 and anti-SSEA-1 markers. mESC colonies and EBs in microfluidic channels demonstrated expression of these ESC antigens after 21 days comparable to mESCs or EBs grown in tissue culture plates (data not shown). EBs also demonstrated differentiation of cells into the three major germ layers mesoderm (smooth muscle actin; SMA), ectoderm (anti-neurofilament; NF) and endoderm (anti-alpha fetoprotein; αFP). These ICC assays show that the EBs under laminar flow conditions are able continue to differentiate into three germ layers.

EB-Microfluidic Chips may be Cryopreserved with Recovery of Function

The fabricated microfluidic cassettes are designed to resist the low temperatures (−196° C.) of cryopreservation by replacing the glass coverslip with 1 mm thick polystyrene plate. EB immobilized microfluidic chips were cultured for 24 h under continuous laminar flow and later cryopreserved according to adopted cryopreservation technique by slow freezing of samples in isopropanol and then stored in liquid nitrogen. After cryopreservation viability of EB was assessed with live/dead assay directly on microfluidic chip (data not shown).

Cryopreserved EB-Microfluidic Chips Recover Steroidogenic Function when Thawed and Cultured

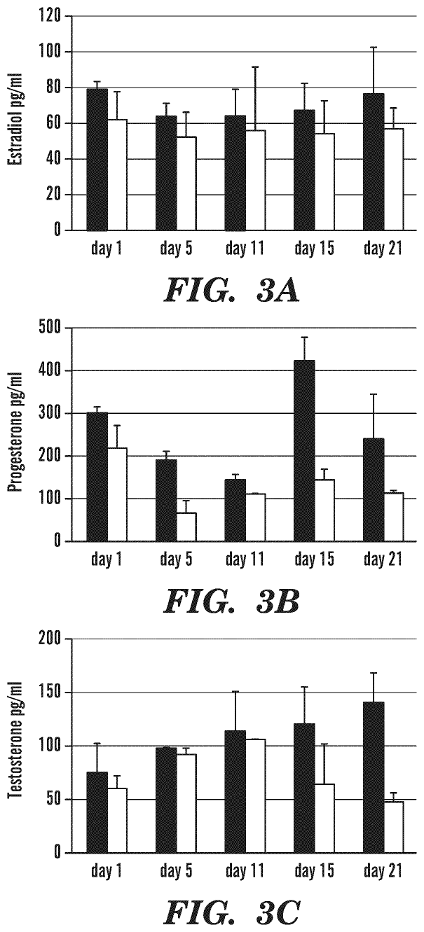

The presence of steroid hormones estradiol, testosterone and progesterone within the conditioned media collected from the dynamic culture of EBs before and after cryopreservation was detected with ELISA analysis. The samples for 24 h period were collected at day 1, 5, 11, 15 and 21, and stored frozen until the analysis. The estradiol level present in the collected samples from non-cryopreserved samples was stable between 64-79 pg/ml for over 21 days period (FIGS. 3A-3C black bars). After the cryopreservation of the EB containing microfluidic chip the estradiol levels decreased, but not to a significant amount (54-62 pg/ml) in 21 days period. Secretion of progesterone in 21 days period showed similar trend for both with and without cryopreservation. The range of progesterone for non-cryopreserved sample was 144 pg/ml and 423 pg/ml for 20 days. The level of secreted testosterone fluctuated between 76 pg/ml and 141 pg/ml in conditions without cryopreservation and 47 pg/ml to 107 pg/ml after the cryopreservation for a 21 days period. The AMH levels for non-cryopreserved channels were detected between 17 pg/ml and 41 pg/ml over 20 days of culture. After the cryopreservation the AMH levels were similar between 19 pg/ml and 45 pg/ml.

### Discussion

Current clinical approaches to regenerative medicine aim to utilize pluripotent stem cells in cell- and gene-based therapies and tissue engineering applications. As the capacity of forming trophoblastic clusters and secrete steroidogenic hormones such as estradiol has been shown the idea to utilize pluripotent stem cells to be used as in vitro agents for secretion of endocrine hormones has emerged^{14,20}. Existing tissue culture methods face specific challenges that include elucidating specific differentiation signals, reproducing in vivo-like differentiation conditions, tissue tolerance, and long-term viability of differentiated tissues in culture²¹. Described herein is an innovative culture system to grow, differentiate, and cryopreserve EBs in a system that allows development of functionally specialized cells and tissues, such as ovarian cells and endocrine tissue.

Among the many goals in the next major phase of stem cell research and regenerative medicine, the ability to generate specific desirable cell types from ESCs and grow organelles, with the hope of eventually generating organs, remain formidable challenges. EBs are formed from embryonic stem cells or induced pluripotent stem cells (iPSCs) and theoretically have the potential to differentiate into any desired cell type such as cardiac cells²², osteogenic and chondrogenic cells²³, neurons²⁴, insulin secreting beta cells²⁵ and steroid hormone secreting cells²⁰. EBs are three dimensional and thus their growth and duration of culture are restricted due to technical limitations such as penetration of media nutrients to the EB's core. Described herein is an improvment on these considerations using a continuous laminar flow system with microfluidic.

### Microfluidic Devices can be Engineered to Mimic the In Vivo Environment

A microfluidic environment provides many advantages in modeling of native-like environments and investigating biological systems^{18,26}. Microenvironments are known to significantly influence the differentiation process of stem cells^{27,28}. For example, the rigidity of the substrate, as well as the gradient of chemokines and growth factors can are important factors in a microenvironment²⁹. Thus, microenvironments can be designed to a desired target tissue or cell type^{25,30} by providing the control of both biological and mechanical stimulation of cells in vitro in a reproducible manner²⁷. The reduced sample size allows costly reagents to be used in smaller quantities and provide a dynamic platform for high throughput screening of chemicals or drugs^{31,32}.

### Development of Steroidogenic Ovarian Cells in a Microfluidic Chip

This study utilizes steroidogenesis and ICC of ovarian antigen expression to begin developing a platform for bioengineering ovarian tissue in a microfluidic module. Steroidogenic cells of the ovary are the primary endocrine tissue of the female reproductive tract and are critical to normal female development, reproductive function and maintenance of a woman's health. In order to be successful at bioengineering such a system, one must attempt to mimic some of the in vivo physiologic parameters of gonadal development and function. In vivo, the primitive gonads develop from intermediate mesoderm from the posterior abdominal wall. This developing tissue is well vascularized and receives adequate perfusion during development and maturation. In the adult ovary, the gonads are supplied by branches of the internal iliac artery as well as a separate ovarian vessel. Thus, the in vivo development and maturation of such endocrine tissue is a dynamic process and favors secretion of synthesized hormones into the vascular bed.

Describe herein is a dynamic flow system that is more similar to the in vivo environment than prior methods by using microfluidic chips¹⁶. While under static flow conditions of cell culture, differentiation of EBs may develop trophoblastic tissue that secretes estradiol, progesterone and human chorionic gonadotropic (hCG)²⁰, demonstrated herein is the continued growth and functional differentiation of endocrine cells in microfluidic chips. Furthermore, similar to recent reports by Lipskind et al. who show expression of ovarian antigens in the differentiating EBs, the EBs cultured on the microfluidic chips also show differentiation of cells that are antigenically similar to ovarian cell types¹³. Taken together, these results indicate that a dynamic flow system using microfluidic chips is indeed a viable option for differentiation of ESC-derived EBs. The EBs grown and differentiated in microfluidic channels show stem cell marker expression concurrent with expression of the makers from the three developmental germ layers, just like what is present in ESC colonies. In addition, to the ovarian lineage marker, AMHR, the expression of follicle-stimulating, hormone receptor (FSHR) is demonstrated. Functional activity of the differentiated tissue is further confirmed with the expression of CYP19A1, showing enzymatic activity for the secretion of estradiol.

### Cell Based Therapies for Hormone Replacement

Traditional drug development and therapeutic approaches to cure diseases are based on mimicking the synthesis of natural molecules or designing biologically active compounds. Although extensive preclinical and clinical trials are performed to address the mechanism of drug activity and ensure the safety of the active compounds, many drugs still have side effects. The activity of a therapeutic agent may vary greatly between the patient populations¹⁰. Developments in molecular biology and genetics bring better understanding of diseases and their potential cures. Current trends in medicine are focusing on personalized approaches specific to the patient, developing customized agents for curing diseases. Described herein is a combination of stem cell biology with bioengineering to formulate a platform for personalized medicine where a dynamic microfluidic system can reflect the natural in vivo environment. In addition, presented herein is a technique where this platform is utilized for synthesizing autologous, steroidogenic hormones which can be cryopreserved for long term storage and thawed on demand. The reduced culture size in a microfluidic system also allows costly reagents to be used in smaller quantities and provide a dynamic platform for high throughput screening of chemicals or drugs^{31,32}. These advancements will be highly useful in cell-based therapies.

### Personalized Medicine

Described herein is a novel application of microfluidics in regenerative medicine, e.g., the development of patient-specific microfluidic treatment modules. The potential applications of such a system are far-reaching for the treatment of other endocrine or neuro-hormonal disorders, such as diabetes with insulin replacement, Parkinson's disease with dopamine replacement or ovarian failure with estrogen and progesterone replacement. For each of these cases, one can employ such microfluidic chips to harvest secreted bioidentical hormone as well as to cryopreserve differentiated EBs within the chip for future use as needed. Similar to medication cartridges that are used today, in the foreseeable future patients can receive autologous personalized treatment using their own iPSCs that are differentiated into the desired secretory cell and grown in individual microfluidic chips.

Previous studies have demonstrated the ability of EBs from human ESCs to produce functional trophoblastic tissue secreting estradiol, progesterone and human chorionic gonadotropin (hCG)²⁰ as well as ovarian granulosa-like cells secreting AMH and FSH³³. With the discovery of iPSCs there has been a heightened excitement in the field of regenerative medicine because a primary obstacle to cell-based therapies has been antigenic matching of tissue. With iPSCs we have options for developing autologous patient specific treatment systems using pluripotent iPSCs that are autologous for the patient³⁴. The unique trait of iPSCs is that they are patient-specific, such that an iPSC line derived from a specific donor will share the same immunological markers as that individual, greatly increasing the odds for success when employed in the context of tissue transplantation or graft³⁴. With regards to hormone replacement therapy, in recent years concerns have been raised by studies such as the Women's Health Initiative (WHI), regarding the risks associated with the use of synthetically produced hormones⁴. Combining the autologous nature of iPSCs with the potential to differentiate iPSC-derived EBs into steroidogenic cells, it is possible to produce bioidentical hormones for the treatment of patients.

### Conclusion

This study demonstrates several novel advancements in regenerative medicine and microfluidics: (i) it is demonstrated herein that microfluidics are a viable system for maintaining EB growth and differentiation; (ii) E2 and P4 are produced at physiologically relevant levels (iii) functionally established microfluidic chips with EBs may be cryopreserved and thawed with restoration of function for use at a later time point. These findings strongly support the utilization of microfluidic chips for future personalized hormone therapies. This approach can be easily adapted to broad clinical applications, such as generation of patient-specific beta islet cells or use as a drug-screening platform for patient-derived tumorigenic cells.

### REFERENCES

1. Howlader N, N. A., Krapcho M, Garshell J, Neyman N, Altekruse S F, Kosary C L, Yu M, Ruhl J, Tatalovich Z, Cho H, Mariotto A, Lewis D R, Chen H S, Feuer E J, Cronin K A (eds). SEER Cancer Statistics Review, 1975-2010, National Cancer Institute, Bethesda, Md. (2013).
2. Siegel, R. et al. Cancer treatment and survivorship statistics, 2012. CA: a cancer journal for clinicians 62, 220-241, doi:10.3322/caac.21149 (2012).
3. Pattimakiel, L. & Thacker, H. L. Bioidentical hormone therapy: clarifying the misconceptions. Cleveland Clinic journal of medicine 78, 829-836, doi:10.3949/ccjm.78a.10114 (2011).
4. Anderson, G. L. et al. Effects of conjugated equine estrogen in postmenopausal women with hysterectomy: the Women's Health Initiative randomized controlled trial. JAMA: the journal of the American Medical Association 291, 1701-1712, doi:10.1001/jama.291.14.1701 (2004).
5. Rossouw, J. E. et al. Risks and benefits of estrogen plus progestin in healthy postmenopausal women: principal results From the Women's Health Initiative randomized controlled trial. JAMA: the journal of the American Medical Association 288, 321-333 (2002).
6. Billeci, A. M., Paciaroni, M., Caso, V. & Agnelli, G. Hormone replacement therapy and stroke. Current vascular pharmacology 6, 112-123 (2008).
7. Bath, P. M. & Gray, L. J. Association between hormone replacement therapy and subsequent stroke: a meta-analysis. Bmj 330, 342, doi:10.1136/bmj.38331.655347.8F (2005).
8. Beral, V., Million Women Study, C., Bull, D., Green, J. & Reeves, G. Ovarian cancer and hormone replacement therapy in the Million Women Study. Lancet 369, 1703-1710, doi:10.1016/S0140-6736(07)60534-0 (2007).
9. Holtorf, K. The bioidentical hormone debate: are bioidentical hormones (estradiol, estriol, and progesterone) safer or more efficacious than commonly used synthetic versions in hormone replacement therapy? Postgraduate medicine 121, 73-85, doi:10.3810/pgm.2009.01.1949 (2009).
10. Moskowitz, D. A comprehensive review of the safety and efficacy of bioidentical hormones for the management of menopause and related health risks. Alternative medicine review: a journal of clinical therapeutic 11, 208-223 (2006).
11. Chlebowski, R. T. et al. Estrogen plus progestin and breast cancer incidence and mortality in postmenopausal women. JAMA: the journal of the American Medical Association 304, 1684-1692, doi:10.1001/jama.2010.1500 (2010).
12. Hamburg, M. A. & Collins, F. S. The path to personalized medicine. The New England journal of medicine 363, 301-304, doi:10.1056/NEJMp1006304 (2010).
13. Lipskind, S., Lindsey, J S, Kiezun, A., Ho, J W K., Gerami-Naini, G. and Anchan, R M. in 60th Annual Meeting of the Society for Gynecologic Investigation (Orlando, Fla., USA, 2013).
14. Golos, T. G., Pollastrini, L. M. & Gerami-Naini, B. Human embryonic stem cells as a model for trophoblast differentiation. Seminars in reproductive medicine 24, 314-321, doi:10.1055/s-2006-952154 (2006).
15. Gothard, D., Roberts, S. J., Shakesheff, K. M. & Buttery, L. D. Controlled embryoid body formation via surface modification and avidin-biotin cross-linking. Cytotechnology 61, 135-144, doi:10.1007/s10616-010-9255-3 (2009).
16. Rizvi, I. et al. Flow induces epithelial-mesenchymal transition, cellular heterogeneity and biomarker modulation in 3D ovarian cancer nodules. Proceedings of the National Academy of Sciences of the United States of America 110, E1974-1983, doi:10.1073/pnas.1216989110 (2013).
17. Gurkan, U. A. et al. Smart interface materials integrated with microfluidics for on-demand local capture and release of cells. Advanced healthcare materials 1, 661-668, doi:10.1002/adhm.201200009 (2012).
18. Gupta, K. et al. Lab-on-a-chip devices as an emerging platform for stem cell biology. Lab on a chip 10, 2019-2031, doi:10.1039/c004689b (2010).
19. Tasoglu, S. et al. Exhaustion of racing sperm in nature-mimicking microfluidic channels during sorting. Small 9, 3374-3384, doi:10.1002/smll.201300020 (2013).
20. Gerami-Naini, B. et al. Trophoblast differentiation in embryoid bodies derived from human embryonic stem cells. Endocrinology 145, 1517-1524, doi:10.1210/en.2003-1241 (2004).
21. Kossowska-Tomaszczuk, K. et al. A novel three-dimensional culture system allows prolonged culture of functional human granulosa cells and mimics the ovarian environment. Tissue engineering. Part A 16, 2063-2073, doi:10.1089/ten.TEA.2009.0684 (2010).
22. Pesl, M. et al. Forced aggregation and defined factors allow highly uniform-sized embryoid bodies and functional cardiomyocytes from human embryonic and induced pluripotent stem cells. Heart and vessels, doi:10.1007/s00380-013-0436-9 (2013).
23. Lee, T. J. et al. Mesenchymal stem cell-conditioned medium enhances osteogenic and chondrogenic differentiation of human embryonic stem cells and human induced pluripotent stem cells by mesodermal lineage induction. Tissue engineering. Part A, doi:10.1089/ten.TEA.2013.0265 (2013).
24. Zhang, Y. et al. Rapid single-step induction of functional neurons from human pluripotent stem cells. Neuron 78, 785-798, doi:10.1016/j.neuron.2013.05.029 (2013).
25. Narayanan, K. et al. Extracellular Matrix-Mediated Differentiation of Human Embryonic Stem Cells: Differentiation to Insulin-Secreting Beta Cells. Tissue engineering. Part A, doi:10.1089/ten.TEA.2013.0257 (2013).
26. Primiceri, E., Chiriaco, M. S., Rinaldi, R. & Maruccio, G. Cell chips as new tools for cell biology—results, perspectives and opportunities. Lab on a chip 13, 3789-3802, doi:10.1039/c31c50550b (2013).
27. Yoshimitsu, R. et al. Microfluidic perfusion culture of human induced pluripotent stem cells under fully defined culture conditions. Biotechnology and bioengineering, doi:10.1002/bit.25150 (2013).
28. Nampe, D. & Tsutsui, H. Engineered micromechanical cues affecting human pluripotent stem cell regulations and fate. Journal of laboratory automation 18, 482-493, doi:10.1177/2211068213503156 (2013).
29. Sun, H., Zhu, F., Hu, Q. & Krebsbach, P. H. Controlling stem cell-mediated bone regeneration through tailored mechanical properties of collagen scaffolds. Biomaterials, doi:10.1016/j.biomaterials.2013.10.054 (2013).
30. Kim, Y. G., Moon, S., Kuritzkes, D. R. & Demirci, U. Quantum dot-based HIV capture and imaging in a microfluidic channel. Biosensors & bioelectronics 25, 253-258, doi:10.1016/j.bios.2009.06.023 (2009).
31. Tasoglu, S., Gurkan, U. A., Wang, S. & Demirci, U. Manipulating biological agents and cells in micro-scale volumes for applications in medicine. Chemical Society reviews 42, 5788-5808, doi:10.1039/c3cs60042d (2013).
32. Wilson, J. L. et al. Single-cell analysis of embryoid body heterogeneity using microfluidic trapping array. Biomedical microdevices, doi:10.1007/s10544-013-9807-3 (2013).
33. Lan, C. W., Chen, M. J., Jan, P. S., Chen, H. F. & Ho, H. N. Differentiation of human embryonic stem cells into functional ovarian granulosa-like cells. The Journal of clinical endocrinology and metabolism 98, 3713-3723, doi:10.1210/jc.2012-4302 (2013).
34. Zhang, J. et al. Differentiation of rat iPS cells and ES cells into granulosa cell-like cells in vitro. Acta biochimica et biophysica Sinica 45, 289-295, doi:10.1093/abbs/gmt008 (2013).

What is claimed herein is:

1. A method of restoring hormonal synthesis in a subject in need thereof, the method comprising:
a. obtaining a stem cell from a first subject;
b. differentiating the cell in vitro into an Embryoid Body; and
c. contacting the Embryoid Body with culture media to differentiate the Embryoid Body into an ovarian cell or tissue.
d. administering the ovarian cell or tissue to a second subject in need of restoration of hormonal synthesis, thereby restoring hormonal synthesis in the second subject.

2. The method of claim 1, wherein the in need of restoration of hormonal synthesis is a subject with or determined to have decreased levels of at least one hormone selected from the group consisting of estradiol, testosterone, and progesterone.

3. The method of claim 1, wherein the ovarian cell obtained in step c. produces at least one hormone selected from the group consisting of estradiol, testosterone, or progesterone.

4. The method of claim 1, wherein the first and second subjects are the same subject.

5. The method of claim 1, wherein the Embryoid Body obtained in step b. is cultured in a microfluidic device coated with an extracellular matrix protein.

6. The method of claim 1, wherein the Embryoid Body obtained in step b. is cultured in suspension.

7. The method of claim 1, wherein the differentiation of the Embryoid Body in step b. into an ovarian cell or tissue occurs in a microfluidic device.

8. The method of claim 1, wherein the culture media of step c. comprises:
Dulbecco's Modified Eagle Medium (DMEM); Fetal Bovine Serum; Alanine; Arginine; Asparagine; Aspartic Acid; Cysteine; Glutamic Acid; Glutamine; Glycine; Proline; Serine; Tyrosine; L-Glutamine; 2-Mercaptoethanol; and Basic Fibroblast Growth Factor.

9. The method of claim 8, wherein the culture media comprises about 15% Fetal Bovine Serum.

10. The method of claim 8, wherein the culture media comprises about 1% Glutamine.

11. The method of claim 8, wherein the culture media comprises about 0.2 mM 2-Mercaptoethanol.

12. The method of claim 8, wherein the culture media comprises about 5 ng/ml Basic Fibroblast Growth Factor.

13. The method of claim 1, wherein the culture media is supplied in a microfluidic device at a flow rate of approximately 1 ul/min for about 21 days.

14. The method of claim 1, wherein the ovarian cell obtained in step c. is a steroidogenic cell.

15. The method of claim 1, wherein the ovarian cell obtained in step c. is a follicular cell.

16. The method of claim 1, wherein the stem cell is an embryonic stem cell or an induced pluripotent stem cell.
